(19) United States
(12) Patent Application Publication
(10) Pub. No.: US 2023/0372264 A1
(43) Pub. Date: Nov. 23, 2023

(54) COMPOSITION FOR THE PREVENTION OF INFECTION BY SARS-COV-2

(71) Applicant: MAGI EUREGIO SCS, Bolzano (BZ) (IT)

(72) Inventors: Matteo BERTELLI, San Felice del Benaco (BS) (IT); Giampietro FARRONATO, Milano (MI) (IT); Marco FARRONATO, Milano (MI) (IT); Gianluca Martino TARTAGLIA, Segrate (MI) (IT); Stefano PAOLACCI, Tivoli (RM) (IT); Kyrylo ANPILOGOV, Valfabbrica (PG) (IT); Elena MANARA, Dro (TN) (IT); Astrit DAUTAJ, Tirana (AL); Paolo Enrico MALTESE, Rovereto (TN) (IT); Kristjana DHULI, Tirana (AL); Mahmut Cerkez ERGOREN, Nicosia (CY); Munis DUNDAR, Melikgazi-Kayseri (TR); Tamer SANLIDAG, Nicosia (CY); Daniele MALACARNE, Pergine (TN) (IT); Irfan S. GUNSEL, Nicosia (CY); Huseyin Kaya SUER, Nicosia (CY); Murat SAYAN, Kocaeli (TR); Gulten TUNCEL, Nicosia (CY); Nazife SULTANOGLU, Nicosia (CY)

(73) Assignee: MAGI EUREGIO SCS, Bolzano (BZ) (IT)

(21) Appl. No.: 18/031,551

(22) PCT Filed: Apr. 7, 2021
(86) PCT No.: PCT/IB2021/052900

§ 371 Date: Apr. 12, 2023

(30) Foreign Application Priority Data
Oct. 13, 2020 (IT) 102020000024118

(51) Int. Cl. A61K 31/05; A61K 31/724; A61K 9/00; A61P 31/14; A61P 31/04

(52) U.S. Cl. CPC A61K 31/05; A61K 31/724; A61K 9/006; A61P 31/14; A61P 31/04

(57) ABSTRACT

A composition includes α-cyclodextrin and hydroxytyrosol and/or their nutraceutically acceptable derivatives, such as salts or esters, in particular for application as a food supplement or medical device, in alternative as a cosmetic mouthwash or traditional medicinal product of plant origin (botanical) or food for special medical purposes. The composition is effective in preventing SARS-CoV-2 infection. The composition is suitable for an application as a spray or mouthwash. A related dispensing device is also described. The composition is also effective in contrasting halitosis in subjects that wear a mask for a prolonged period.

### TECHNICAL FIELD

The invention concerns a composition to strengthen the defenses in the context of the COVID-19 pandemic caused by the severe acute respiratory syndrome coronavirus 2, abbreviated as SARS-CoV-2, and to help to effectively prevent an infection from SARS-CoV-2. The composition can be administered orally, preferably in the form of a spray, but alternatively it can be administered nasally, in this regard the composition can be applied, for example, through a food supplement, a medical device, a cosmetic mouthwash, a traditional herbal medicinal product (botanical), a food for special medical purposes, and this preferably in the form of a spray or in liquid format.

### STATE OF THE ART

The SARS-CoV-2 virus resides in the mucous membranes: The SARS-CoV-2 virus is able to attack, at first instance, the cells of the respiratory mucous membranes, where it may reside for two days before spreading to the lower respiratory tract, as evidenced for SARS-CoV virus in macaques (SARS-CoV virus is very similar to SARS-CoV-2 virus for the cell tropism) [1]. Human-to-human transmission of the virus occurs through coughing, sneezing, inhalation of droplets and direct contact with the mouth, nose and eyes with contaminated hands [2]. The infection has an average incubation period of 6.4 days and a basic reproduction of 2.24-3.58 [3].

All respiratory viruses with envelope, such as SARS-CoV-2, bind to specific cell membrane receptors that facilitate their entry into the cell itself. Cellular infection occurs through two phases i) binding of the virus to the cell through surface viral proteins attaching to cell membrane receptors and ii) membrane fusion induced by conformational changes in the fusion proteins [4]. Specifically, the SARS-CoV-2 virus uses the Spike (S) protein present in the envelope to bind the cellular ACE2 receptor located in the lipid rafts. ACE2 cuts the Spike protein proteolytically, this induces a series of mechanisms that lead to viral endocytosis. The ACE2 receptor binds to the Spike protein only when it is localized in lipid rafts [5]. Lipid rafts are membrane subdomains characterized by a high content of sphingolipids, cholesterol and proteins [6]. Numerous studies have revealed that lipid rafts are important during coronavirus viral infection. Studies have shown that drug-mediated elimination of cholesterol inhibits the entry of human coronaviruses [7, 8] into host cells. Furthermore, lipid rafts have been reported to be crucial for SARS-CoV entry into Vero E6 cells [9]. Lipid rafts can be destroyed by cell cholesterol depletion via methyl-β-cyclodextrin (MβCD) [10]. In fact, it has been found that the integrity of the lipid raft is necessary for the productive infection of SARS-CoV (virus very similar to SARS-CoV-2). Treatment with MβCD relocates the ACE2 receptor outside the lipid raft and this treatment is able to inhibit SARS-CoV infectivity by 90% [9].

The β-cyclodextrins are able to bind cholesterol, whereas α-cyclodextrins, with a smaller internal cavity, cannot bind cholesterol, but are able to bind membrane phospholipids and sphingolipids. In particular, it has been shown that cyclodextrins can influence the composition of the cell membrane as evidenced by a study published in 2007; in this case, it was found that β-cyclodextrin could reduce the cholesterol content of the plasma membrane [11]. Methyl-β-cyclodextrin reduces the amount of cholesterol in cell membranes and can inhibit the attack of coronaviruses on host cells [10, 12]. Exposure to MβCD can also lead to the redistribution of cholesterol between lipid rafts and non-raft regions [11]. In vitro cellular models have shown that cholesterol depletion by MβCD halves the number of ACE2 receptor bonds with viral S-glycoproteins [5]. Some studies have also shown that treatment with MβCD slightly and dose-dependently reduces the expression of ACE2 in the cell membrane, reducing the infectivity of coronaviruses [13].

Research aimed at combating the SARS-CoV-2 virus is currently concentrated in the pharmaceutical fields, while efforts in prevention are often aimed at improving the body's defenses. Besides vaccination, fully satisfactory solutions have not been identified so far.

### DISCLOSURE OF THE INVENTION

The scope of the invention is to effectively enhancing the defenses for the prevention of SARS CoV-2 infection. A further scope of the invention is to find an effective composition for the aforementioned prevention that can be applied orally by spray. Another scope of the invention is to identify such a composition consisting of nutraceutical components.

In a first aspect of the invention, the scope is achieved by a composition which includes (as active ingredients):

(a) α-cyclodextrin,
(b) hydroxytyrosol
and/or their derivatives, such as nutraceutical and/or pharmaceutically acceptable salts or esters

In particular, the composition is a composition for an application as a food supplement or with a medical device. In alternative embodiments, it is a composition for application as medical device, cosmetic mouthwash, traditional medicine of vegetable origin (botanical), or food for special medical purposes.

In its application as medical device, the composition preferably further comprises hydroxypropyl methylcellulose.

According to IUPAC, hydroxytyrosol can also be called 4-(2-hydroxyethyl)-1,2-dihydroxybenzene (n. CAS 10597-60-1). Both α-cyclodextrin and hydroxytyrosol are already approved as novel food by European legislation.

The hypothesis of the inventors is the possibility to take advantage of the lipid rafts disruption by modifying their composition and influencing the subsequent endocytosis of SARS-CoV-2 or other viruses that share the same mechanism of lipid-raft mediated endocytosis, being this protection non specific.

Results of a recent study support the hypothesis that α-cyclodextrins can act in this sense: it has been proven that α-cyclodextrin is able to replace sphingolipids and phospholipids in the outer layer of the plasma membrane with exogenous lipids in mammalian cells, thus acting in a bidirectional way [14]. Furthermore, thanks to their structure, α-cyclodextrins preferably bind saturated fatty acids, which, together with cholesterol, make up lipid rafts. Therefore, it is plausible that α-cyclodextrins can modify the composition of lipid rafts, destabilizing them. Furthermore, α-cyclodextrin has already been used with a different mechanism of action in which they reduced the concentration of serum phospholipids in equilibrium with the membrane phospholipids. This reduced serum concentration causes a malfunction of the cellular pathways on the membrane exploited by the virus for endocytosis in the cell [15]. In another work by Wittkowski it was highlighted how the α-cyclodextrins, being smaller, are not able to deplete cholesterol, but phospholipids. For example, hydroxypropyl-α-cyclodextrin is twice as effective as hydroxypropyl-p-cyclodextrin against the migration of human breast cancer cells involving an endocytosis/exocytosis mechanism [16]. In addition, the effects of α-cyclodextrin as a virucidal molecule have been described. In this case, α-cyclodextrin was modified with mercaptoundecane sulfonic acid, to mimic heparan sulfate and block heparan sulfate-dependent viruses such as HSV-2 [17].

Hydroxytyrosol, on the other hand, is a phenolic compound with antioxidant properties obtained from olive extracts. It is authorized as a nutraceutical compound and as a food supplement under both European and US legislation. In vivo, hydroxytyrosol exhibits anti-inflammatory and anti-viral activity. In vivo a bacteriostatic activity has been noted, too. Hydroxytyrosol has antiviral effects on the influenza virus, in fact hydroxytyrosol appears to cause morphological changes in the H9N2 influenza virus pretreated with hydroxytyrosol, without affecting neuraminidase or haemagglutinin activity [18]. Hydroxytyrosol has also been shown to be an inhibitor of fusion (lipid raft mediated endocytosis [19]) and integration of HIV-1. Hydroxytyrosol binds to the viral protein gp41, interfering with the fusion of the viral membrane with the cellular membrane. Hydroxytyrosol shows a dose-dependent inhibition, without detectable cytotoxicity [20]. In addition, hydroxytyrosol also binds to HIV-1 integrase. Hydroxytyrosol binds to region II of the integrase active site, inhibiting its activity in a dose-dependent manner [21]. Hydroxytyrosol is also able to induce an anti-inflammatory effect, decreasing the levels of pro-inflammatory cytokines such as IL-6 and TNF-α in animal models [22]. Finally, in mouse models it was found that hydroxytyrosol is able to lower serum lipids in rats fed a diet rich in cholesterol [23], thus being able to act indirectly on the composition of the plasma membrane. Hydroxytyrosol interacts directly with the plasma membrane and localizes at the level of the hydrophilic heads. This could modify the physico-chemical properties of the membrane important for the process of lipid raft mediated endocytosis [24]. Therefore, for hydroxytyrosol there are generally anti-viral activities, but to the knowledge of the inventors, so far it has never been used in the SARS-CoV-2 context. In addition, hydroxytyrosol can have bacteriostatic effects [25, 26] and this property, in a preferred embodiment of the invention, could be exploited to counteract, contemporarily with the prevention according to the invention, halitosis due to prolonged use of facial masks. Bacteriostatic effects have been shown in strains of Candida sp., Staphylococcus aureus, Streptococcus spp. with a growth reduction of 40% [27]. The inventors have discovered, through bioinformatics approach that, hydroxytyrosol, in combination with α-cyclodextrins, has promising activities in the prevention of SARS-CoV-2 infection wherein the cyclodextrin serves as a vehicle for hydroxytyrosol and both compete in the occupation of the attack sites on the cell membranes of the host cells. Furthermore, using the bioinformatics tool D3DOCKING [28], specialized for the study of the interaction of small molecules that interfere with the life cycle of SARS-CoV-2 in humans, it was possible to identify the presence of possible direct interactions between hydroxytyrosol and α-cyclodextrin with the viral protein Spike and with the human proteins TMPRSS2 and ACE2 (FIGS. 3 and 4).

Molecular docking experiments with AutodockVina-based YASARA software [29] were performed to confirm the possibility of interaction between α-cyclodextrin and hydroxytyrosol with the spike protein and ACE2. In vitro experiments on VeroE6 animal cell lines were also performed to confirm the virostatic properties of the alpha-cyclodextrin and hydroxytyrosol compounds [30].

A preferred formulation of the invention therefore provides for the application of the composition in the prevention of SARS-CoV-2 infection. Prevention takes place in a specific and effective way. At the same time, the combination can obviously have other virucidal or therapeutic effects, such as those already known for hydroxytyrosol. In particular, the above mentioned bacteriostatic effects can be exploited. A further preferred embodiment of the invention, involves the simultaneous application of the composition in combating halitosis due to prolonged mask use. Hypothetical is the application of the composition also only for contrasting halitosis in subjects who wear a mask for prolonged times.

Advantageously, the components of the composition are present in effective quantities, that is, effective in the prevention of SARS-CoV-2 infection, as shown by the observational and interventional studies conducted.

Preferably, the composition is present in the form of an aqueous solution in which hydroxytyrosol is present in a concentration of about 3.80-11.40% (m/m) and α-cyclodextrin is present in a concentration of about 0.20-0.60% (m/m). The specific solution mentioned above has a density of 1.1 g/ml. Also lower concentrations of hydroxytyrosol (from 1.00 to 11.40% (m/m)) in the combination have been shown to be effective in contrasting halitosis. In this case, the density varies from 1.0 to 1.1 g/ml. Other solvents or excipients are conceivable, as it is conceivable to add further components usual in the sector, such as flavorings.

The composition according to the invention, thanks to the hypothesized operating mechanism, can have oral application in the form of a spray. Alternatively, the application can be nasally. Nasal or oral applications as a spray or also in liquid form are feasible, in particular as a mouthwash, which is particularly suitable for combating halitosis.

Thus, oral or nasal applications by spray or in liquid formulation are conceivable, for example as a food supplement, medical device, cosmetic mouthwash, traditional medicinal product of plant origin (botanical), food for special medicinal purposes.

In a very advantageous variant of the invention, oral application or oral administration by means of a spray or as a mouthwash, involves applying the composition to the oropharyngeal mucosa. In another very advantageous embodiment of the invention, the oral application via spray, or alternatively as a mouthwash, or the nasal administration foresees an application of the composition on the nasal or oropharyngeal mucosa. This application and the following action of the composition as described above presents an opportunity to strengthen the defenses through the non-specific but effective reduction against SARS-CoV-2 of the process of lipid raft mediated endocytosis and consequently of prevention [2], also taking advantage of the fact that the virus probably stops and replicates exponentially for two days on the mucous membranes before its diffusion in the lower airways [31], thus interfering with endocytosis, the biological cycle of the virus is limited, made by exocytosis, endocytosis and multiplication. Furthermore, as already explained above, in its formulation as a mouthwash, the composition has a bacteriostatic effect that reduces the growth in the oral cavity of bacteria responsible for halitosis from prolonged use of the mask [27].

In an Advanced Statistics for Drug and Diet Exploration, Repurposing, and Approval (ASDERA) study [32] there is a different approach of using α-cyclodextrin alone to inhibit the entry of SARS-CoV-2, which is based on oral administration and intestinal absorption of the active ingredient.

Advantageously, application on the oropharyngeal mucosa is an application as a spray that performs its action topically. Alternatively, the application on the oropharyngeal mucosa is an application as a mouthwash that performs its action topically. Alternatively, the application is done on the nasal mucosa, in particular as a spray.

According to a variant of the invention, the application involves repeated applications, even without time limitations, preferably twice a day, preferably after careful oral hygiene, each for a total of 0.5 ml of the aqueous solution as specified above. Preferably, each 0.5 mL dose is applied with four sprays from a spray dispensing device, each spray delivering approximately 0.125 mL of solution.

The inventors therefore propose for the first time the use of the combination of hydroxytyrosol and α-cyclodextrin to strengthen non-specific defenses through the reduction of the endocytosis process and consequent reduction of the risk of SARS-CoV-2 infection based on the principle that α-cyclodextrin inhibits lipid raft mediated endocytosis and hydroxytyrosol has a destabilizing action on the plasma membrane. Bioinformatics studies show how both hydroxytyrosol and α-cyclodextrin interact with the proteins necessary for viral endocytosis.

A second aspect of the invention concerns a dispensing device which comprises
(i) a container containing the composition according to the invention;
(ii) a spray head for dispensing the composition.

The market offers various systems and medical devices useful for this purpose, such as classic aerosol dispensers or spray cans. In particular for the aqueous solution described above, the spray head is advantageously adapted to deliver doses after activation, the multiple of which, preferably four, corresponds to 0.5 ml of said aqueous solution. Alternatively, the multiple corresponds to 1 mL of said aqueous solution. It thus allows the application of correct amounts of the active ingredients. Alternatively, eight spray doses are foreseen which correspond to 0.5 or 1 mL of solution.

The device according to the invention can also comprise the composition according to the invention in the form of a mouthwash.

A further aspect of the invention concerns a method for the treatment or the prevention of SARS-CoV-2 infection which involves the application of the composition according to the invention in particular on the oropharyngeal mucosa, or in alternative on the nasal mucosa. It is therefore the use of the composition according to the invention for the prevention of SARS-CoV-2 infection.

It is therefore the use of the composition according to the invention for the prevention of SARS-CoV-2 infection. Preferably, the application takes place in concentrations (dosages) and according to the administration plan illustrated above.

A final aspect of the invention relates to a process for the production of a composition referred to in which α-cyclodextrin and hydroxytyrosol and/or their nutraceutical and/or pharmaceutically acceptable derivatives are mixed. Preferably, the two components are dissolved in water, in particular in the amounts indicated above.

The features described for one aspect of the invention can be transferred mutatis mutandis to the other aspects of the invention.

The variants of the invention described above achieve the intended scopes of the invention. The composition according to the invention is effective in preventing SARS-CoV-2 infection, has no side effects, shows no cytotoxic effects and contains nutraceutical components.

The aforementioned scopes and advantages will be further highlighted during the description of a preferred embodiment example of the invention given by way of non-limiting example.

Variants of the invention are the subject of dependent claims. The description of the preferred embodiment example of the composition according to the invention and of the method of application is given by way of non-limiting example with reference to the attached drawings.

In practice, the materials used, as well as the dimensions, numbers and shapes, provided they are compatible with the specific use if not otherwise specified, may be any according to requirements. Furthermore, all the details can be replaced by other technically equivalent elements.

### DESCRIPTION OF A PREFERRED EMBODIMENT EXAMPLE

### Brief Description of the Figures

FIG. 1 shows the results of a cytotoxicity test of a spray containing α-cyclodextrin and hydroxytyrosol on the human cell line Caco-2.

FIG. 2 shows the results of a cytotoxicity test of a spray containing α-cyclodextrin and hydroxytyrosol on the human cell line HepG2.

FIG. 3 shows the potential binding sites of hydroxytyrosol with the proteins ACE2, TMPRSS2 and Spike.

FIG. 4 shows the potential binding sites of α-ciclodestrina with the proteins ACE2, TMPRSS2 and Spike.

FIG. 5 shows growth inhibition, ranging from 20 to 80%, in cultures of Candida albicans (A), Staphylococcus aureus (B) and a mix of bacteria (Streptococcus sp., Staphylococcus sp. and Neisseria sp.) from throat swab (C), grown in standard culture medium (below) and in standard culture medium with the addition of mouthwash according to the invention (above).

FIG. 6 shows the reduction of DNA amplification by PCR of bacterial (A) and fungal (B) DNA after using the mouthwash (samples 7, 8 and 9) in individuals who have worn the mask continuously for 8 hours, while it is highlighted a greater presence of bacteria and fungi after using the mask for 8 hours without using mouthwash.

### In Vitro and In Vivo Experimentation for the Safety and Efficacy Profiles for the Prevention of SARS-CoV-2 Infection

In an embodiment example of the invention, an aqueous solution of α-cyclodextrin and hydroxytyrosol is produced in which hydroxytyrosol is present in a concentration of 3.80-11.40% (m/m), whereas α-cyclodextrin has a concentration of 0.20-0.60% (m/m). The solution is transferred into a spray dispensing device and is thus usable as a spray.

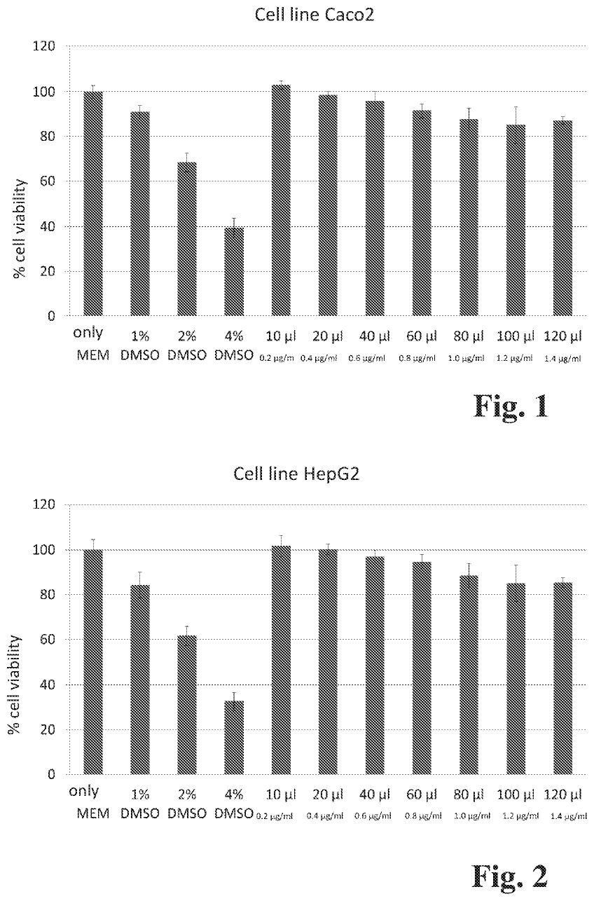

In vitro, the spray of the invention is not cytotoxic as observed in two human cell lines, Caco2 and HepG2 (FIGS. 1 and 2) at increasing concentrations (MEM=Modified Eagle's Medium; DMSO=dimethylsulfoxide).

The composition has antioxidant properties as showed in a ORAC assay (Oxygen Radical Absorbance Capacity), that demonstrate an antioxidant capacity of 1,247.43±4.05 μmol TE/ml (micromoles of Trolox® (TE) per milliliter of the sample). Interestingly, the antioxidant properties of the human milk of mothers fed with a Mediterranean diet has an antioxidant power of 584.16+29.51 μmol TE/mi.

The spray was tested on 87 volunteers negative for COVID-19 and with different clinical characteristics (table 1) in a observational study without controls. None of these individuals after using the spray for a week has shown any side effects or interactions with drugs taken for other diseases.

The volunteers took two doses per day, each dose corresponds to four spray doses for a total of 0.5 ml of solution (3.80% (m/m) of hydroxytyrosol and 0.20% (m/m) of α-cyclodextrin.

Since the participants were all residents of Lombardy and given that 1% of the population was infected at the peak period [33], at least one affected patient was expected among the participants, but at the end of the study they were all negative for the virus.

TABLE 1

| | | |
|---|---|---|
| Mean age ± standard deviation (Min-Max) | 52.6 ± 17.6 | (24-86) |
| Males/Females (% males) | 45/42 | (51.7%) |
| Smoker Yes/No (% Yes) | 36/51 | (41.4%) |
| Comorbidity Yes/No (% Yes) | 36/51 | (41.4%) |
| Diabetes | 12/51 | (23.5%) |
| Obesity | 9/51 | (17.6%) |
| Hypercolesterolemia | 11/51 | (21.6%) |
| Hypertension | 17/51 | (33.3%) |
| Cardiovascular diseases | 11/51 | (21.6%) |
| Dyslipidemia | 4/51 | (7.8%) |
| Pollen allergy | 1/51 | (2%) |
| Anemia | 1/51 | (2%) |
| Pharmacological treatments Yes/No (% Yes) | 44/43 | (50.6%) |
| ACE inhibitors | 13/44 | (29.5%) |
| β-blockers | 11/44 | (25%) |
| Anticoagulants | 9/44 | (20.5%) |
| Hypoglycemic agents | 9/44 | (20.5%) |
| Statins | 8/44 | (18.2%) |
| Mean weight (kg) ± SD (Min-Max) | 78.6 ± 12.6 | (55-107) |
| Mean height (m) ± SD (Min-Max) | 1.73 ± 0.1 | (1.53-1.92) |
| Mean BMI (Body Mass Index) ± | 26.1 ± 3.7 | (17-37.7) |
| SD (Min-Max) | | |
| Type of exposure to virus (continuous/ | 36/51 | (41.4%) |
| occasional)* | | |
| Place of exposure (home/workplace) | 28/58 | (32.5%) |

| | |
|---|---|
| Withdrawal from the study | 0% |
| Side effects | 0% |

*Continuous exposure means health workers in continuous contact with COVID-19 patients or cohabiting with sick subjects. By occasional, we mean any contact with a casual and not protracted positive subject as it can be in public transport or at a restaurant.

The results shown in Table 1 are a first clue to the effectiveness of the spray in the prevention of COVID-19. The use of the spray by people with a greater risk of contracting the infection, such as healthcare workers or family members of affected patients, gives a strong indication of the reduction of the risk of contracting the infection. The percentage of subjects who can acquire the infection after contact at risk is not certain, but it is estimated that it can range from a minimum of 30% of contacts up to 80%. Wang et al. demonstrated that 47 (30%) of 155 close contacts tested positive with SARS-CoV-2, indicating that the secondary transmission rate among family contacts of SARS-CoV-2 patients was 30% [34]. Other recently published studies highlight how it is possible to find, in the context of different family clusters, much higher percentages, which reach a maximum of 80% of contacts [35]. A difference of 20% can therefore be considered between non-prophylaxed subjects (in which we assume a 30% acquisition of infection) and those who have been prophylaxed (in which we assume a 10% acquisition of infection).

The invention achieved the aim of proposing a composition with nutraceutical active ingredients that are effective in preventing SARS-CoV-2 infection and that do not have side effects or signs of cytotoxicity. The proposed oral administration allows in particular the local (topical) action of the two components in the oropharyngeal mucosa, made possible by the particular combination of α-cyclodextrin and hydroxytyrosol.

In the executive phase, further modifications or executive variants not described may be made to the composition and other related aspects, subject of the invention. Should such modifications or variants fall within the scope of the following claims, they must all be considered protected by this patent.

In collaboration with the “Near East” University of Nicosia (Cyprus), the spray was tested on 90 SARS-CoV-2 negative volunteers at the start of the study to further evaluate its safety and effectiveness in preventing infection. After 30 days of using the spray, no volunteers was tested positive for the swab and/or serological test (Table 2).

TABLE 2

| Mean age ± SD (SD = standard | 36.3 ± 11.0 | (20-76) |
|---|---|---|
| deviation) (Min-Max) | | |
| Males/Females (% males) | 44/46 | (49%) |
| Smoker Yes/No (% Yes) | 33/57 | (36.7%) |
| Comorbidity Yes/No (% Yes) | 13/77 | (14.4%) |
| Diabetes | 3 | |
| Hypercolesterolemia | 2 | |
| Hypertension | 4 | |
| Cardiovascular diseases | 2 | |
| Thyroid diseases | 3 | |
| Food allergy | 1 | |
| Favism | 1 | |
| Obesity | 1 | |
| Dyslipidemia | 1 | |
| Pharmacological treatments Yes/No (% Yes) | 12/78 | (13.3%) |
| ACE inhibitors | 4 | |
| β-blockers | 1 | |
| Proton pump inhibitors | 2 | |
| Hypoglycemic agents | 3 | |
| Thyroid hormone analogues | 2 | |
| Anti-vertigo | 1 | |
| Colchicine | 1 | |
| Mean weight (kg) ± SD (Min-Max) | 75.7 ± 17.2 | (48-126) |
| Mean height (m) ± DS (Min-Max) | 1.70 ± 0.1 | (1.50-1.96) |
| Mean BMI (Body Mass Index) ± | 25.3 ± 5.3 | (18.1-37.9) |
| SD (Min-Max) | | |
| Type of exposure to virus (continuous/ | 46/44 | (51.1%) |
| occasional)* | | |
| Place of exposure (home/workplace) | 0/90 | (0%) |

| | |
|---|---|
| Withdrawal from the study | 0% |
| Side effects | 0% |

*Continuous exposure means health workers in continuous contact with COVID-19 patients or cohabiting with sick subjects. By occasional, we mean any contact with a casual and not protracted positive subject as it can be in public transport or at a restaurant.

Finally, the spray was tested in a small cohort of 6 swap positive patients after real time PCR assay for SARS-CoV-2. The biology of the virus reminds us that swap positivity does not always correspond to the clinical manifestation of the disease. Therefore, the use of the spray in the absence of a clinical diagnosis of the disease can still be useful. Of these 6 patients, 2 used the spray. It is interesting to note that the two treated patients became negative after 3 days, while the 4 untreated patients became negative after 6 days (Table 5), this data further supports the role of the spray in strengthening the non-specific defenses against viruses that attack the organism through lipid raft mediated endocytosis, and in particular against the SARS-CoV-2 virus.

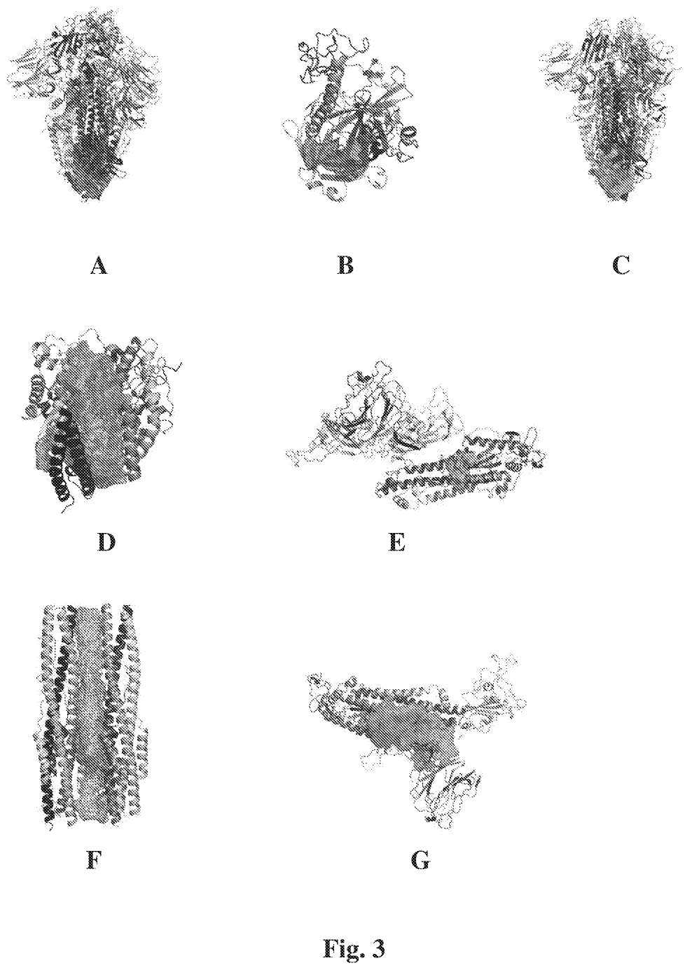

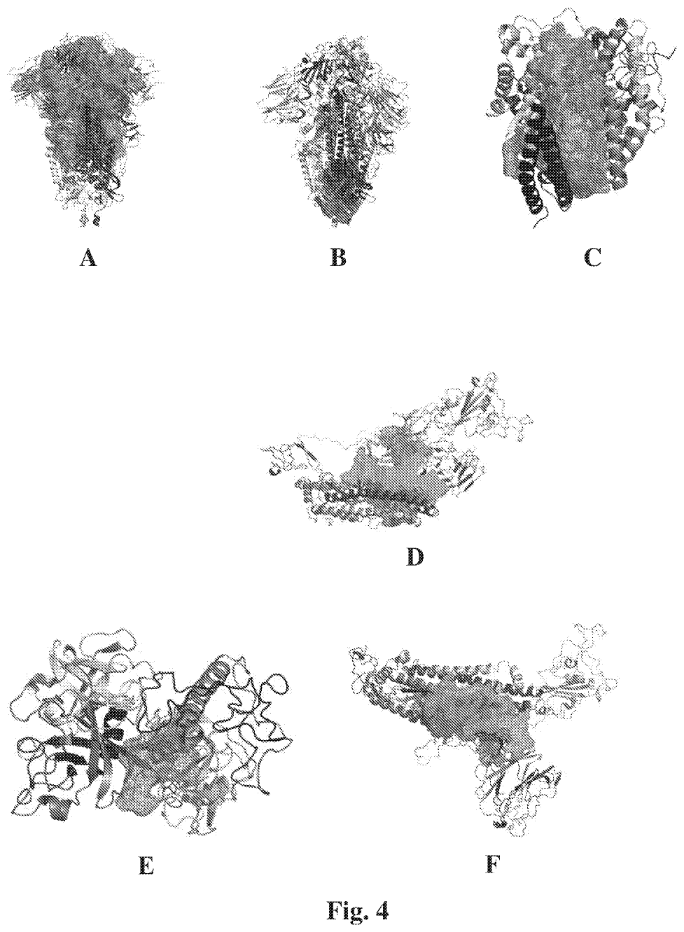

FIG. 3 illustrates the potential binding sites of hydroxytyrosol with the proteins ACE2, TMPRSS2 and Spike, while FIG. 4 illustrates the potential binding sites of α-cyclodextrin with ACE2, TMPRSS2 and Spike proteins.

As shown above, with the bioinformatic tool D3DOCKING [28], specialized for the study of the interactions of small molecules that interfere with the biological cycle of SARS-CoV-2 in humans, it was possible to find the presence of possible direct interactions between hydroxytyrosol and α-cyclodextrin with the viral protein Spike and with the human proteins TMPRSS2 and ACE2. Table 3 shows the details relevant for the FIG. 3, table 4 refers to FIG. 4.

TABLE 3

| | Target | | | | | | |
|---|---|---|---|---|---|---|---|
| | (complete | | | | | Template | |
| Panel | name) | State | Score | ID protein | Pocket | PDB_ID* | Organism |
| A | Spike protein | open | −7.21 | QHD43416.1 | 2 | 6vyb | SARS- |
| | (S) | | | | | | CoV-2 |
| B | Transmembrane | | −6.87 | O15393 | 4 | — | human |
| | serine protease 2 | | | | | | |
| | (TMPRSS2) | | | | | | |
| C | Spike protein | closed | −6.82 | QHD43416.1 | 2 | 6vxx | SARS- |
| | (S) | | | | | | CoV-2 |
| D | Angiotensin | | −6.54 | P59594 | 1 | 1r42 | human |
| | converting | | | | | | |
| | enzyme 2 | | | | | | |
| | (ACE2) | | | | | | |
| E | Spike protein | open | −6.5 | QHD43416.1 | 5 | 5x5b | SARS- |
| | (S) | | | | | | CoV-2 |
| F | Spike protein, | | −6.24 | / | 1 | 6lxt | SARS- |
| | S2 subunit | | | | | | CoV-2 |
| G | Spike protein | closed | −6.18 | QHD43416.1 | 1 | 5x58 | SARS- |
| | (S) | | | | | | CoV-2 |

*PDB = protein data bank

TABLE 4

| | Target | | | | | | |
|---|---|---|---|---|---|---|---|
| | (complete | | | | | Template | |
| Panel | name) | State | Score | ID protein | Pocket | PDB_ID* | Organism |
| A | Spike protein | closed | −11.88 | QHD43416.1 | 1 | 6vxx | SARS- |
| | (S) | | | | | | CoV-2 |
| B | Spike protein | open | −11.06 | QHD43416.1 | 2 | 6vyb | SARS- |
| | (S) | | | | | | CoV-2 |
| C | Angiotensin | | −10.92 | P59594 | 1 | 1r42 | human |
| | converting | | | | | | |
| | enzyme 2 | | | | | | |
| | (ACE2) | | | | | | |
| D | Spike protein | open | −9.71 | QHD43416.1 | 1 | 5x5b | SARS- |
| | (S) | | | | | | CoV-2 |
| E | Transmembrane | | −9.42 | O15393 | 2 | — | human |
| | serine protease 2 | | | | | | |
| | (TMPRSS2) | | | | | | |
| F | Spike protein | closed | −8.88 | QHD43416.1 | 1 | 5x58 | SARS- |
| | (S) | | | | | | CoV-2 |

*PDB = protein data bank

TABLE 5

| | | | | | Pharmaco- | | | | | |
|---|---|---|---|---|---|---|---|---|---|---|
| | | | | Comor- | logical | Weight | Height | | Type of | Place of |
| Patient | Sex | Age | Smoker | bidity | treatments | (kg) | (m) | BMI | exposure | exposure |
| 1 | F | 47 | Yes | No | No | 57 | 1.64 | 21.2 | Contin- | Work |
| | | | | | | | | | uous | (physician) |
| 2 | M | 50 | No | Obe- | No | 95 | 1.70 | 32.9 | Contin- | Work |
| | | | | sity | | | | | uous | (businessman, |
| | | | | | | | | | | often traveling) |
| 3 | M | 57 | No | No | No | 89 | 1.82 | 26.8 | Contin- | Work |
| | | | | | | | | | uous | (politician) |
| 4 | M | 42 | No | No | No | 80 | 1.82 | 24.1 | Contin- | Work |
| | | | | | | | | | uous | (driver) |
| 5 | F | 37 | No | No | No | 61 | 1.64 | 22.6 | Occa- | Work |
| | | | | | | | | | sional | (personal |
| | | | | | | | | | | trainer) |
| 6 | F | 36 | No | No | No | 58 | 1.62 | 22.1 | Contin- | Work (personal |
| | | | | | | | | | uous | assistant of |
| | | | | | | | | | | politician) |

| | SARS-CoV-2 RT-qPCR | | | | SARS-CoV-2 | | | | |
|---|---|---|---|---|---|---|---|---|---|
| | (viral load) | | | | serologic test | | | | |
| | Days | | | | Day 0 | | Day 30 | | Symp- |
| Patient | 0 | 5 | 10 | 15 | IgG | IgM | IgG | IgM | toms |
| 1 | + | − | − | − | + | + | + | | High |
| | (2.5 × 10⁵) | | | | | | | | fever |
| 2 | + | − | − | − | + | + | + | | Backpain |
| | (2.5 × 10⁹) | | | | | | | | |
| 3 | + | + | − | − | + | + | + | | Asymp- |
| | (2.5 × 10⁵) | (2.5 × 10⁴) | | | | | | | tomatic |
| 4 | + | + | − | − | + | + | + | | Asymp- |
| | (2.5 × 10²) | (2.5 × 10²) | | | | | | | tomatic |
| 5 | + | + | − | − | | | | | Mild |
| | (2.5 × 10⁹) | (2.5 × 10⁴) | | | | | | | fever |
| 6 | + | + | − | − | | | | | Asymp- |
| | (2.5 × 10⁵) | (2.5 × 10²) | | | | | | | tomatic |

* Continuous exposure means health workers in continuous contact with COVID-19 patients or cohabiting with sick subjects. By occasional, we mean any contact with a casual and not protracted positive subject as it can be in public transport or at a restaurant.

Molecular docking experiments were performed using Autodock Vina-based YASARA software [29] to confirm the possibility of interaction between α-cyclodextrin and hydroxytyrosol with the spike protein and ACE2. The results showed that α-cyclodextrin can bind to both the viral spike protein and the host receptor protein ACE2 with a significantly higher binding affinity than hydroxytyrosol for the same proteins. The binding energies of α-cyclodextrin and hydroxytyrosol to ACE2 were 7.91 kcal/mol and 6.10 kcal/mol, respectively, while the corresponding binding energies to the spike protein were 6.40 kcal/mol and 6.41 kcal/mol, respectively. In conclusion, both compounds could inhibit the two targets by virtue of their significant binding affinities. These compounds can also form physical bonds with each other, and the resulting complex is predicted to bind efficiently to both proteins. Compared to its components, the binding energy of the complex is significantly higher for the ACE2 receptor and similar to the separate compounds for the spike protein [36]. Finally, in vitro experiments were performed on animal cell lines, VeroE6. These experiments showed that at 0.8 μM a solution containing α-cyclodextrin and hydroxytyrosol is not cytotoxic and is able to inhibit viral replication (Table 6) [30].

TABLE 6

| | A-cyclodextrin (CD) + Hydroxytyrosol (HT) | | | | | | | | | |
|---|---|---|---|---|---|---|---|---|---|---|
| | | Viability, % of the | | | | Viral replication (NP, ng/ml) | | | | |
| | Concentration, | control | | | Concentration, | | | % of the | | |
| | (μM) | Mean | SD | CC₅₀ | (μM) | Mean | SD | control | IC₅₀ | |
| | Control | 100.0 | 0.0 | (μM) | Control | 1140 | 103 | 100 | (μM) | SI₅₀ |
| CD + | 100 | 11.5 | 0.0 | 1.7 | 100 | Toxic | — | — | 1 | 1.7 |
| HT | 20 | 18.4 | 0.5 | | 20 | Toxic | — | — | | |
| | 4 | 7.5 | 0.5 | | 4 | Toxic | — | — | | |
| | 0.8 | 92.9 | 17.6 | | 0.8 | 772 | 18 | 68 | | |
| | 0.16 | 87.7 | 21.9 | | 0.16 | 1056 | 149 | 93 | | |
| | 0.032 | 84.4 | 21.8 | | 0.032 | 856 | 95 | 75 | | |

### Further Clinical Studies

The clinical data of the 225 subjects (heterogeneous in age, sex, comorbidities and drug use, with no significant differences from the general population) who used the spray for 30 days and those who did not use it are included in Tables 7 and 8. All participants were followed up for 30 days until the last spray administration. Although all the participants were at increased risk of becoming infected with SARS-CoV-2 due to their occupation, none of the users of the spray showed any side effects and none of them acquired the infection even though while administering the spray they have certainly been in contact with at least 7 of their colleagues, who contracted the virus by attending the same work environment as 12 users of the spray [30]. The clinical data for the subjects that used the spray are listed in table 7.

TABLE 7

| Characteristics | Values | |
|---|---|---|
| Participants, n | 149 | |
| Mean age ± SD (range) | 37 ± 11.45 | (20-76) |
| Males/Females (% males/% females) | 74/75 | (49.7%/50.3%) |
| Smoker Yes/No (% Yes) | 63/86 | (42.3%/57.7%) |
| Comorbidity, n (%) | 22/127 | (14.8%) |
| Diabetes | 4 | (2.7%) |
| Hypercolesterolemia | 4 | (2.7%) |
| Hypertension | 9 | (6.0%) |
| Cardiovascular diseases | 2 | (1.7%) |
| Thyroid diseases | 3 | (2.6%) |
| Food allergy | 1 | (0.8%) |
| Favism | 1 | (0.8%) |
| Arrhythmia | 1 | (0.8%) |
| Obesity | 2 | (1.7%) |
| Dyslipidemia | 3 | (2.0%) |
| Ulcerative colitis | 1 | (0.8%) |
| Crohn disease | 1 | (0.8%) |
| Hypoglycemia | 1 | (0.8%) |
| Pharmacological treatments, n (%) | 15/134 | (10%/90%) |
| β-blockers | 1 | (0.8%) |
| Proton pump inhibitors | 2 | (1.7%) |
| Hypoglicemic agents | 4 | (3.5%) |
| Synthetic thyroid hormones | 2 | (1.7%) |
| Colchicine | 1 | (0.8%) |
| Angiotensin II receptor inhibitors | 3 | (2.6%) |
| α-blockers | 1 | (0.8%) |
| Anti-hypertensive | 1 | (0.8%) |
| Diuretics | 1 | (0.8%) |
| Anti-vertigo | 1 | (0.8%) |
| Immunosuppressants | 1 | (0.8%) |
| Mean weight (kg) ± SD (Range) | 75 ± 16.67 | (48-126) |
| Mean height (m) ± SD (Range) | 1.70 ± 0.1 | (1.50-1.96) |
| Mean BMI ± SDS (Range) | 26.0 ± 7.24 | (17.6-37.9) |

| | |
|---|---|
| Type of exposure to virus (continuous/ | 149/0 |
| occasional) | |
| Place of exposure (home/workplace) | 0/149 |
| Withdrawal from the study | 0 |
| Side effects | 0 |
| SARS-CoV-2 infection acquisition | 0 |

The clinical data of subjects that did not use the spray are listed in the following table 8.

TABLE 8

| Characteristics | Values | |
|---|---|---|
| Participants, n | 76 | |
| Mean age ± SD (range) | 31.7 ± 11.45 | (20-76) |
| Males/Females (% males/% females) | 39/37 | (51.3%/48.7%) |
| Smoker Yes/No (% Yes) | 28/48 | (36.8%/63.2%) |
| Comorbidity, n (%) | 15/61 | (19.7%) |
| Diabetes | 2 | (2.6%) |
| Hypertension | 3 | (3.9%) |
| Cardiovascular diseases | 1 | (1.3%) |
| Thyroid diseases | 2 | (2.6%) |
| Obesity | 4 | (5.3%) |
| Ulcerative colitis | 2 | (2.6%) |
| Chronic migraine | 1 | (1.3%) |
| Dizziness | 1 | (1.3%) |
| Chronic pharyngitis | 1 | (1.3%) |
| Pharmacological treatments, n (%) | 6/70 | (7.9%) |
| Synthetic thyroid hormones | 2 | (2.6%) |
| Angiotensin II receptor inhibitors | 1 | (1.3%) |
| Anti-vertigo | 1 | (1.3%) |
| Anti-inflammatory agents | 2 | (2.6%) |
| Pain killers | 1 | (1.3%) |
| Anticoagulants | 1 | (1.3%) |
| Mean weight (kg) ± SD (Range) | 72.9 ± 16.67 | (48-126) |
| Mean height (m) ± SD (Range) | 1.70 ± 0.1 | (1.50-1.96) |
| Mean BMI ± SD (Range) | 24.9 ± 7.24 | (17.6-37.9) |

| | |
|---|---|
| Type of exposure to virus (continuous/ | 76/0 |
| occasional) | |
| Place of exposure (home/workplace) | 0/76 |
| Withdrawal from the study | 0 |
| Side effects | 0 |
| SARS-CoV-2 infection acquisition | 0 |

### In Vitro and In Vivo Testing of the Application of Hydroxytyrosol and α-Cyclodextrin in the Form of Mouthwash to Counteract Halitosis Due to Prolonged Use of Facial Mask

The possible inhibition of the growth of microorganisms by a mouthwash containing the composition according to the invention was evaluated: hydroxytyrosol and α-cyclodextrin. The following microorganisms were isolated from throat swabs collected by sterile swabs and cultured:
1) Candida albicans (isolated from a lingual swab)
2) Staphylococcus aureus (isolated from pus culture)
3) Mix of bacteria from throat swab containing Streptococcus sp., Staphylococcus sp. and Neisseria sp.

The microorganisms were inoculated into a standard liquid medium called Brain Heart Infusion (BHI) agar (Liofilchem).

Media with the microorganisms were incubated at 37° C. for 4 hours in order to reach the logarithmic growth phase. Four tubes containing 2 ml of BHI medium and 4 tubes containing 1 ml of BHI and 1 ml of mouthwash were prepared. In each tube 100 μl of liquid medium from logarithmic cultures were inoculated. In order to obtain 1) a tube with 2 ml of BHI medium inoculated with 100 μl of medium containing Candida albicans and a tube with 1 ml of BHI medium+1 ml of mouthwash inoculated with 100 μl of medium containing Candida albicans; 2) a tube with 2 ml of BHI medium inoculated with 100 μl of medium containing Staphylococcus aureus and a tube with 1 ml of BHI medium+1 ml of mouthwash inoculated with 100 μl of medium containing Staphylococcus aureus; 3) a tube with 2 ml of BHI medium inoculated with 100 μl of medium containing a mix of bacteria from throat swab and a tube with 1 ml of BHI medium+1 ml of mouthwash inoculated with 100 μl of medium containing a mix of bacteria from throat swab.

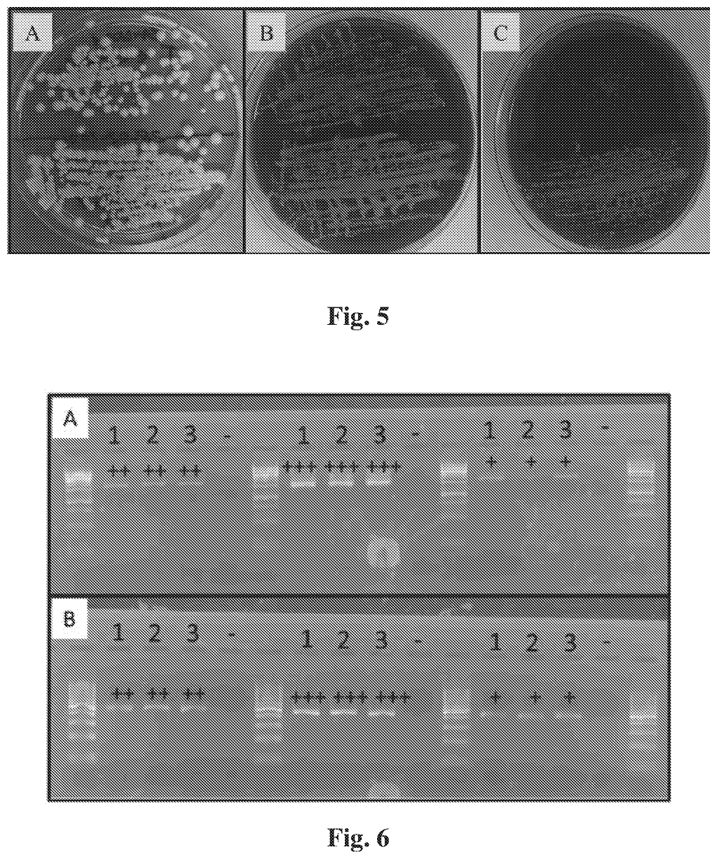

After an overnight incubation of all cultures at 37° C., 1 μl was inoculated on solid media in Petri dishes divided into two parts: one with BHI medium and one with BHI medium and mouthwash. Sabouraud Dextrose Agar (Biolife) was used for C. albicans, while Columbia Blood Agar (Biolife) for the other cultures. After incubation at 37° C. overnight, a growth difference highlighted in FIG. 5 was confirmed, FIG. 5 illustrates the inhibition of the growth that oscillates between 20 and 80%, in the cultures of Candida albicans (A), Staphylococcus aureus (B) and a mix of bacteria (Streptococcus sp., Staphylococcus sp. and Neisseria sp.) from throat swap (C), cultivated in standard culture medium (below) and in standard culture medium withy adding a mouthwash according to the invention (above).

From the experimental data obtained, an evident inhibition of growth was observed, oscillating between 20 and 80%, in particular in the culture from throat swab. In fact, the mixed flora of Streptococcus sp., Staphylococcus sp. and Neisseria sp. is the most similar to the real microbial flora of the oral cavity [27].

In a further experiment to evaluate the effect of the mouthwash, DNA was extracted from saliva in three volunteers: without using the mask, after using the mask for 8 hours, after they had used the mask for 8 hours and then used the mouthwash. Then the extracted DNA was amplified with universal primers (for example Universal 16S rRNA bacterial primers 27F and Universal Panfungal ITS) for the amplification of bacterial and fungal DNA as described in the references [37,38], to evaluate possible differences in microbial growth with or without the use of the facial mask and with or without the use of the mouthwash. It has been shown that there is an increase in bacterial and fungal growth when wearing the mask for 8 hours, while when the volunteers wore the mask for 8 hours and then used the hydroxytyrosol and α-cyclodextrin-based mouthwash, a reduction in the presence of bacteria and fungi was found, even compared to those who did not use the mask for 8 hours (FIG. 6) [27].

FIG. 6 shows ladders of DNA, wherein A (ladder 1 kb) refers to the primer Universal 16S rRNA bacterial primers 27F with a fragment length of 1440 bp and B (ladder 100 bp) to the primer Universal Panfungal ITS with a fragment length of 500 bp. From the left to the right, results without mask, with a use of the mask for 8 hours and with a use of the mask together with an application of the mouthwash according to the invention can be noted.

A 10 days study has been performed. In the first 5 days, 12 volunteers did not use the mouthwash, while in the remaining 5 days the same volunteers used the mouthwash. As a basic assumption it was considered that all volunteers use toothbrushes and toothpaste after each meal and wear the mask for 8 hours a day. After that, these volunteers were given a questionnaire to assess whether they had experienced halitosis at the beginning of the study and if they perceived improvements after the use of the mouthwash at the end of the study. At the end of the study, a strong tendency was found towards a reduction of subjects with halitosis from 9 to 2, with gingival inflammation from 6 to 2 and of the oral cavity from 8 to 2, and dryness of the oral cavity from 5 to 3 [27] (Table 9 lists the answers to the questionnaire concerning the use of the mouthwash.).

TABLE 9

| | | Reduction in |
|---|---|---|
| Disturbance | Value | percentage |
| Halitosis | Before: 9/12 | 78% |
| | After: 2/12 | |
| Gingival inflammation | Before: 6/12 | 67% |
| | After: 2/12 | |
| Oral cavity inflammation | Before: 8/12 | 75% |
| | After: 2/12 | |
| Dry oral cavity | Before: 5/12 | 40% |
| | After: 3/12 | |

### BIBLIOGRAPHIC REFERENCES

[1] L. Liu, Q. Wei, K. Nishiura, J. Peng, H. Wang, C. Midkiff, X. Alvarez, C. Qin, A. Lackner, Z. Chen; Spatiotemporal interplay of severe acute respiratory syndrome coronavirus and respiratory mucosal cells drives viral dissemination in rhesus macaques. Mucosal Immunol. 2016; 9:1089-1101. (doi:10.1038/mi.2015.127).
[2] A. A. Caruso, A. Del Prete, A. I. Lazzarino, R. Capaldi, L. Grumetto: Might hydrogen peroxide reduce the hospitalization rate and complications of SARS-CoV-2 infection? Infect. Control Hosp. Epidemiol. 2020:1-2. (doi:10.1017/ice.2020.170).
[3] C.-C. Lai, T.-P. Shih, W.-C. Ko, H.-J. Tang, P.-R. Hsueh; Severe acute respiratory syndrome coronavirus 2 (SARS-CoV-2) and coronavirus disease-2019 (COVID-19): The epidemic and the challenges. Int. J. Antimicrob. Agents 2020; 55(3):105924. (doi:10.1016/j.ijantimicag.2020.105924).
[4] M. Marsh, A. Helenius; Virus entry: Open sesame. Cell 2006; 124:729-740. (doi:10.1016/j cell.2006.02.007).
[5] J. Glende, Chr. Schwegmann-Wessels, M. Al-Falah, S. Pfefferle, X. Qu, H. Deng, Chr. Drosten, H. Y. Naim, G. Herder; Importance of cholesterol-rich membrane microdomains in the interaction of the S protein of SARS-coronavirus with the cellular receptor angiotensin-converting enzyme 2. Virology 381 (2008) 215-221 (doi.org/10.1016/j.virol.2008.08.026).
[6] K. Simons, E. Ikonen; Functional rafts in cell membranes. Nature 387 (1997) 569-572 (doi:10.1038/42408).
[7] R. Nomura, A. Kiyota, E. Suzaki, K. Kataoka, Y. Ohe, K. Miyamoto, T. Senda, T. Fujimoto; Human Coronavirus 229E Binds to CD13 in Rafts and Enters the Cell through Caveolae. J. Virol. 78 (2004) 8701-8708 (doi:10.1128/JVI.78.16.8701-8708.2004)
[8]E. B. Thorp, T. M. Gallagher; Requirements for CEACAMs and Cholesterol during Murine Coronavirus Cell Entry. J. Virol. 78 (2004) 2682-2692 (doi:10.1128/JVI.78.6.2682-2692.2004).
[9] Y. Lu, D. X. Liu, J. P. Tam; Lipid rafts are involved in SARS-CoV entry into Vero E6 cells. Biochem. Biophys. Res. Commun. (2008) 369(2):344-349 (doi:10.1016/j.bbrc.2008.02.023).
[10] H. Guo, M. Huang, Q. Yuan, Y. Wei, Y. Gao, L. Mao, L. Gu, Y. W. Tan, Y. Zhong, D. Liu, Sh. Sun; The Important Role of Lipid Raft-Mediated Attachment in the Infection of Cultured Cells by Coronavirus Infectious Bronchitis Virus Beaudette Strain. PLoS ONE (2017) 12(1): e0170123 (doi:10.1371/journal.pone.0170123).
[11] R. Zidovetzki, I. Levitan; Use of cyclodextrins to manipulate plasma membrane cholesterol content: evidence, misconceptions and control strategies. Biochim. Biophys. Acta (2007) 1768(6): 1311-1324 (doi:10.1016/j.bbamem.2007.03.026).
[12] M. Baglivo, M. Baronio, G. Natalini, T. Beccari, P. Chiurazzi, E. Fulcheri, P. Petralia, S. Michelini, G. Fiorentini, G. A. Miggiano, A. Morresi, G. Tonini, M. Bertelli; Natural small molecules as inhibitors of coronavirus lipid-dependent attachment to host cells: a possible strategy for reducing SARS-COV-2 infectivity? Acta Biomed. 2020; Vol. 91 (1): 161-164
[13] G.-M. Li, Y.-G. Li, M. Yamate, Sh.-M. Li, K. Ikuta; Lipid rafts play an important role in the early stage of severe acute respiratory syndrome-corona-virus life cycle. Microbes Infect. (2007) 9:96-102 (doi: 10.1016/j.micinf.2006.10.015).
[14] G. Li, J. Kim, Zh. Huang J. R. St. Clair, D. A. Brown, E. London; Efficient replacement of plasma membrane outer leaflet phospholipids and sphingolipids in cells with exogenous lipids. PNAS 113 (49) (2016) 14025-14030 (doi.org/10.1073/pnas.1610705113).
[15] K. M. Wittkowski; A Novel Formulation of α-Cyclodextrin for Use as a Natural Anti-Aging ‘Intermittent Fasting Mimetic’. (2019) (https://app.box.com/s/j3of6yxbd7dn8fp4guce003b7awokai1).
[16] K. M. Wittkowski, Chr. Dadurian, M. P. Seybold, H. S. Kim, A. Hoshino, D. Lyden; Complex polymorphisms in endocytosis genes suggest alpha-cyclodextrin as a treatment for breast cancer. PLoS ONE (2018) 13(7): e0199012 (doi.org/10.1371/journal.pone.0199012).
[17] S. T. Jones, V. Cagno, M. Janedek, D. Ortiz, N. Gasilova, J. Piret, M. Gasbarri, D. A. Constant, Y. Han, L. Vukovic, P. Král, L. Kaiser, S. Huang, S. Constant, K. Kirkegaard, G. Boivin, F. Stellacci, C. Tapparel; Modified cyclodextrins as broad-spectrum antivirals. Sci. Adv. 2020; 6(5):eaax9318 (doi:10.1126/sciadv.aax9318).
[18] K. Yamada, H. Ogawa, A. Hara, Y. Yoshida, Y. Yonezawa, K. Karibe, V. Bui Nghia, H. Yoshimura, Y. Yamamoto, M. Yamada, K. Nakamura, K. Imai; Mechanism of the antiviral effect of hydroxytyrosol on influenza virus appears to involve morphological change of the virus. Antiviral Res. 2009; 83(1):35-44 (doi.org/10.1016/j.antiviral.2009.03.002).
[19] S. T. Yang, V. Kiessling, J. A. Simmons, J. M. White, L. K. Tamm; HIV gp41-mediated membrane fusion occurs at edges of cholesterol-rich lipid domains. Nat. Chem. Biol. (2015); 11(6):424-31 (doi: 10.1038/nchembio.1800).
[20] S. Lee-Huang, P. L. Huang, D. Zhang, J. W. Lee, J. Bao, Y. Sun, Y.-T. Chang, J. Zhang, P. L. Huang; Discovery of small-molecule HIV-1 fusion and integrase inhibitors oleuropein and hydroxytyrosol: Part I. fusion [corrected] inhibition. Biochem. Biophys. Res. Commun. (2007) 354(4):872-8 (doi: 10.1016/j.bbrc.2007.01.071).
[21] S. Lee-Huang, Ph. L. Huang, D. Zhang, J. W. Lee, J. Bao, Y. Sun, Y.-T. Chang, J. Zhang, P. L. Huang; Discovery of small-molecule HIV-1 fusion and integrase inhibitors oleuropein and hydroxytyrosol: part II. integrase inhibition. Biochem. Biophys. Res. Commun. (2007) 354(4):879-84 (doi: 10.1016/j.bbrc.2007.01.058).
[22] M. J. Ramirez-Expósito, J. M. Martinez-Martos. Anti-Inflammatory and Antitumor Effects of Hydroxytyrosol but Not Oleuropein on Experimental Glioma In Vivo. A Putative Role for the Renin-Angiotensin System. Biomedicines 2018; 6(1): 11 (doi:10.3390/biomedicines6010011).
[23] H. Jemai, I. Fki, M. Bouaziz, Z. Bouallagui, A. El Feki, H. Isoda, S. Sayadi; Lipid-Lowering and Antioxidant Effects of Hydroxytyrosol and Its Triacetylated Derivative Recovered from Olive Tree Leaves in Cholesterol-Fed Rats. J. Agric. Food Chem. (2008); 56(8):2630-2636 (doi: 10.1021/jf072589s).
[24] S. Lopez, B. Bermudez, S. Monserrat-de la Paz, S. Jaramillo, L. M. Varela, A. Ortega-Gomez, R. Abia, F. J. G. Muriana; Membrane composition and dynamics: A target of bioactive virgin olive oil constituents. Biochim. Biophys. Acta 1838 (2014) 1638-1656 (doi:10.1016/j.bbamem.2014.01.007).
[25] J. Thielmann, S. Kohnen, C. Hauser; Antimicrobial activity of Olea europaea Linné extracts and their applicability as natural food preservative agents. Int. J. Food Microbiol. 2017; 251:48-66. (doi:10.1016/j.ijfoodmicro.2017.03.019).
[26] J. Wei, S. Wang, D. Pei, L. Qu, Y. Li, J. Chen, D. Di, K. Gao; Antibacterial activity of hydroxytyrosol acetate from olive leaves (Olea europaea L.). Nat. Prod. Res. 2018; 32(16):1967-1970. (doi:10.1080/14786419.2017.1356830).
[27] Naureen Z, Capodicasa N, Anpilogov K, Coatto M, Paolacci S, Manara E, Dautaj A, Dhuli K, Camilleri G, Bertelli M. Prevention of the proliferation of oral pathogens due to prolonged mask use based on alpha-cyclodextrin and hydroxytyrosol mouthwash. Submitted to the European Reviews for Medical and Pharmacological Sciences
[28] https://www.d3pharma.com/D3Targets-2019-nCoV/D3Docking/index.php
[29] O. Trott O, A. J. Olson; AutoDock VINA: Improving the speed and accuracy of docking with a new scoring function, efficient optimization and multithreading. J. Comput. Chem. 2010; 31:455-461. (doi:10.1002/jcc.21334).
[30] Paolacci S, Ergoren M C, De Forni D, Manara E, Poddesu B, Cugia G, Dhuli K, Camilleri G, Tuncel G, Suer H K, Sultanoglu N, Sayan M, Dundar M, Gunsel I S, Coatto M, Dautaj A, Sanlidag T, Connelly S T, Tartaglia G M, Bertelli M. In vitro and clinical studies on the efficacy of alpha-cyclodextrin and hydroxytyrosol against SARS-CoV-2 infection. Submitted to European Review for Medical and Pharmacological Sciences
[31] L. Liu, Q. Wei, K. Nishiura, J. Peng, H. Wang, C. Midkiff, X. Alvarez, C. Qin, A. Lackner, Z. Chen; Spatiotemporal interplay of severe acute respiratory syndrome coronavirus and respiratory mucosal cells drives viral dissemination in rhesus macaques. Mucosal. Immunol. 2016; 9:1089-1101 (doi: 10.1038/mi.2015.127).
[32] http://www.asdera.com/sars-nutrition.html
[33] https://lab24.ilsole24ore.com/coronavirus/.
[34] Zh. Wang, W. Ma, X. Zheng, G. Wu, R. Zhang; Household transmission of SARS-CoV-2. J. Infec. 81 (2020) 179-182 (doi:10.1016fj.jinf.2020.03.040).
[35] K. Danis, O. Epaulard, Th. Bénet, A. Gaymard, S. Campoy, E. Botelho-Nevers, M. Bouscambert-Duchamp, G. Spaccaferri, F. Ader, A. Mailles., Boudalaa, V. Tolsma, J. Berra, S. Vaux, E. Forestier, C. Landelle, E. Fougere, A. Thabuis, Ph. Berthelot, R. Veil, D. Levy-Bruhl, Chr. Chidiac, B. Lina, B. Coignard, Chr. Saura; Cluster of Coronavirus Disease 2019 (COVID-19) in the French Alps, February 2020. Clinical Infectious Diseases (2020); 71(15):825-832 (doi:10.1093/cid/ciaa424).
[36] Paolacci S, Kiani A K, Shree P, Tripathi D, Tripathi Y B, Tripathi P, Tartaglia G M, Farronato M, Farronato G, Connelly S T, Beccari T, Ceccarini M R, Coatto M, Ergoren M C, Sanlidag T, Dautaj A, Bertelli M. Scoping review on the role and interactions of hydroxytyrosol and alpha-cyclodextrin in lipid-raft-mediated endocytosis of SARS-CoV-2 and bioinformatic molecular docking studies. Submitted to European Review for Medical and Pharmacological Sciences
[37] Miller C S, Handley K M, Wrighton K C, Frischkorn K R, Thomas B C, Banfield J F. Short-read assembly of full-length 16S amplicons reveals bacterial diversity in subsurface sediments. PLoS One 2013; 8(2):e56018. doi:10.1371/journal.pone.0056018
[38] Meason-Smith C, Edwards E E, Older C E, Branco M, Bryan L K, Lawhon S D, Suchodolski J S, Gomez G, Mansell J, Hoffmann A R. Panfungal polymerase chain reaction for identification of fungal pathogens in formalin-fixed animal tissues. Vet Pathol 2017; 54(4):640-648. doi:10.1177/0300985817698207
[39] Thayer R E. The biopsychology of mood and arousal. New York: Oxford University Press; 1989
[40] Országhová Z, Waczulíková I, Burki C, Rohdewald P, Ďuračková Z. An effect of oak-wood extract (Robuvit®) on energy state of healthy adults—A pilot study. Phytother Res 2015; 29(8): 1219-1224

1. A composition comprising:
(a) α-cyclodextrin, and
(b) hydroxytyrosol
and/or their nutraceutically acceptable derivatives, such as salts or esters.

2. The composition according to claim 1 for application as a food supplement or with a medical device.

3. The composition according to claim 1 for application as medical device, cosmetic mouthwash, traditional medicinal product of plant origin (botanical) or food for special medical purposes, in particular as a mouthwash.

4. The composition according to the claim 1 for use in the prevention of the SARS-CoV-2 infection.

5. The composition according to claim 4 for the contemporaneous reduction of halitosis for prolonged use of a mask.

6. The composition according to claim 4, wherein the two components are present in an effective amount.

7. The composition according to claim 6, wherein said composition is present in the form of an aqueous solution wherein hydroxytyrosol is present in a concentration of about 3.80-11.40% (m/m) and said α-cyclodextrin is present in a concentration of about 0.20-0.60% (m/m).

8. The composition according to claim 4, wherein said use takes place by oral administration.

9. The composition according to claim 4, wherein said use takes place by nasal administration.

10. The composition according to claim 8, wherein aid administration is an administration on the oropharyngeal mucosa.

11. The composition according to claim 9, wherein said administration is an administration on the nasal mucosa.

12. The composition according to claim 10, wherein said administration on the oropharyngeal or nasal mucosa is an application as liquid or spray.

13. The composition according to claim 10, wherein said administration is an application as a spray and foresees repeated administrations, preferably two per day, each of about 0.5 ml of said aqueous solution and wherein said composition is present in the form of an aqueous solution wherein hydroxytyrosol is present in a concentration of about 3.80-11.40% (m/m) and said α-cyclodextrin is present in a concentration of about 0.20-0.60% (m/m).

14. The composition according to claim 12, wherein said administration is an application as a mouthwash and foresees repeated administrations, preferably two per day, each of about 5 ml of said aqueous solution and wherein said composition is present in the form of an aqueous solution wherein hydroxytyrosol is present in a concentration of about 3.80-11.40% (m/m) and said α-cyclodextrin is present in a concentration of about 0.20-0.60% (m/m).

15. A mouthwash comprising a composition comprising:
(a) α-cyclodextrin, and
(b) hydroxvtyrosol
and/or their nutraceutically acceptable derivatives, such as salts or esters.

16. A dispensing device comprising
(i) a container containing a composition comprising:
(a) α-cyclodextrin, and
(b) hydroxvtyrosol
and/or their nutraceutically acceptable derivatives, such as salts or esters;
(ii) a spray head for dispensing said composition.

17. The dispensing device according to claim 16, that wherein the spray head is adapted to dispense doses after activation, the multiple of which, preferably four or eight, corresponds to about 0.5 or 1 ml of said aqueous solution.
